SEQUENCE LISTING (10 sequences)

SEQ ID NO: 1 - DNA, 22 bp, Artificial Sequence
  Chemically-synthesized sequencing primer
atgacagtgatagcacatcaga

SEQ ID NO: 2 - DNA, 22 bp, Artificial Sequence
  Chemically-synthesized sequencing primer
attgttaccattgccattactg

SEQ ID NO: 3 - DNA, 18 bp, Artificial Sequence
  Chemically-synthesized sequencing primer
cagtgaggaagatggcca

SEQ ID NO: 4 - DNA, 21 bp, Artificial Sequence
  Chemically-synthesized sequencing primer
cttggcagtcattgtcatctt

SEQ ID NO: 5 - DNA, 19 bp, Artificial Sequence
  Chemically-synthesized sequencing primer
cggatcctgaccaaaaacc

SEQ ID NO: 6 - DNA, 20 bp, Artificial Sequence
  Chemically-synthesized sequencing primer
tcatgatgtccgtggtcaat

SEQ ID NO: 7 - DNA, 21 bp, Artificial Sequence
  Chemically-synthesized sequencing primer
gactgtggttaccgtcatggc

SEQ ID NO: 8 - DNA, 23 bp, Artificial Sequence
  Chemically-synthesized sequencing primer
acttggtttttcataacagcgga

SEQ ID NO: 9 - DNA, 20 bp, Artificial Sequence
  Chemically-synthesized sequencing primer
gcaccgtcaaggctgagaac

SEQ ID NO: 10 - DNA, 19 bp, Artificial Sequence
  Chemically-synthesized sequencing primer
tggtgaagacgccagtgga

(19) United States
(12) Patent Application Publication
(10) Pub. No.: US 2019/0290550 A1
(43) Pub. Date: Sep. 26, 2019

(54) METFORMIN-CONTAINING DENTAL MATERIALS

(71) Applicants: Huakun XU, Frederick, MD (US); Su-Ping WANG, Halethorpe, MD (US); Mark A. REYNOLDS, Baltimore, MD (US); Abraham SCHNEIDER, Owings Mills, MD (US)

(72) Inventors: Huakun XU, Frederick, MD (US); Su-Ping WANG, Halethorpe, MD (US); Mark A. REYNOLDS, Baltimore, MD (US); Abraham SCHNEIDER, Owings Mills, MD (US)

(73) Assignee: UNIVERSITY OF MARYLAND, Baltimore, Baltimore, MD (US)

(21) Appl. No.: 16/360,394

(22) Filed: Mar. 21, 2019

Related U.S. Application Data
(60) Provisional application No. 62/646,468, filed on Mar. 22, 2018.

(51) Int. Cl. A61K 6/00; A61K 31/155; A61K 31/14; A61K 33/38; A61K 33/42

(52) U.S. Cl. CPC A61K 6/0067; A61K 31/155; A61K 6/0023; A61K 6/0073; A61K 6/0088; A61K 33/38; A61K 33/42; A61K 6/0091; A61K 31/14

(57) ABSTRACT

The present invention provides metformin-containing dental materials that can be widely applied in a variety of dental applications. The metformin-containing dental materials of the invention include metformin-containing dental primers, metformin-containing dental adhesives, metformin-containing dental resins, metformin-containing dental composites, metformin-containing dental bonding systems and the like, as well as metformin-containing dental cements, metformin-containing dental sealants, metformin-containing dental bases and metformin-containing dental liners.

### BACKGROUND

Dental caries is the most common infectious disease in humans worldwide [1]. Dental resins are widely used in tooth cavity restorations [2-6]. In cases of deep cavities, liners and bases are often used beneath a restoration, which include calcium hydroxide-based, glass ionomer-based, resin-based and zinc phosphate-based materials [7,8]. Liners and bases can help provide protection for the dental pulp, prevent microleakage under the restoration, inhibit bacterial growth, reduce postoperative sensitivity, and release fluoride into the tooth [7,9]. Besides the need for liners and bases, the success of deep carious cavity treatment also depends on the self-repairing ability of the host pulpal cells, especially odontoblasts [10].

Odontoblasts, as a layer of palisade cells lining the interface between the dental pulp and inner dentin, are specialized cells capable of synthesizing not only primary dentin during early tooth development, but also secondary dentin throughout the life span of the tooth [11]. Odontoblasts synthesize the matrix of type I collagen, and actively participate in its mineralization by secreting proteoglycans and non-collagenous proteins that are implicated in the nucleation and growth of the mineral phase [12]. They also participate in the maintenance of the pulp vitality by synthesizing tertiary dentin (reparative dentin). The primary odontoblasts could be destroyed when exposed to deep caries or severe dental injury [13]. In deep cavities with the remaining dentin thickness (RDT)<0.25 mm, the number of odontoblasts decreased by 23% [10,14]. However, dental pulp stem cells (DPSCs), as precursor undifferentiated mesenchymal cells, can differentiate into odontoblasts and have the ability to regenerate dentin/pulp-like complexes in response to carious stimuli [15,16]. Therefore, in order to heal teeth and maintain a viable pulp, the development of bioactive liner/base materials that can promote odontoblastic differentiation and reparative dentin formation would be beneficial.

Metformin (1,1-dimethylbiguanide hydrochloride), an anti-diabetic biguanide drug, is widely used to treat type 2 diabetes mellitus (T2DM) by controlling blood sugar levels [17]. The mechanism of action of metformin is not clearly understood, but is generally thought to be suppressing hepatic gluconeogenesis. The gut microbiota could serve as a major determinant of metformin action and metformin could alter the gut microbiome of individuals with type 2 diabetes [18]. Several studies indicated that metformin has an osteogenic effect by promoting the differentiation of mesenchymal stem cells (MSCs) and preosteoblasts [19-21]. Indeed, metformin induced the differentiation and mineralization of preosteoblasts into osteoblasts via activation of the AMP-activated kinase (AMPK) signaling pathway [22,23]. A recent study showed that metformin promoted the osteogenic differentiation and mineralization of induced pluripotent stem cell-derived MSCs (iPSC-MSCs) via liver kinase B1 (LKB1)/AMPK pathway [24]. Hence, metformin could help enhance bone and periodontal regeneration in diabetic patients. DPSCs share similar gene expression profiles and differentiation capabilities to other MSCs [25-27] and it was recently demonstrated that metformin induced odontoblastic differentiation of DPSCs [28].

The development of metformin-containing dental materials that release metformin and stimulate DPSCs for mineral synthesis and dentin production would be beneficial for use in the many dental applications. The present invention is directed to the development of such materials and other important goals.

### SUMMARY

Disclosed herein are metformin-containing dental materials that can be widely applied to a variety of dental products and applications. The invention is thus directed to metformin-containing dental materials.

In particular, the metformin-containing dental materials of the invention are those that comprise (i) metformin in an amount of about 0.01% to about 40% of the mass of the dental material and (ii) one or more resins in a combined amount of about 20% to about 99.99% of the mass of the dental material.

The metformin-containing dental materials of the invention include, but are not limited to, metformin-containing dental resins, metformin-containing dental adhesives, metformin-containing dental cements, metformin-containing dental composites, metformin-containing dental primers, metformin-containing dental sealants, metformin-containing dental bases, metformin-containing dental liners, metformin-containing dental bonding agents, metformin-containing dental bonding systems, metformin-containing dental tooth caries restorations, metformin-containing dental varnishes, and metformin-containing dental coatings.

In a first aspect of the invention, the metformin-containing dental material is a metformin-containing dental resin comprising metformin and one or more resins in the amounts indicated above, namely (i) metformin in an amount of about 0.01% to about 40% of the mass of the dental resin and (ii) one or more resins in a combined amount of about 20% to about 99.99% of the mass of the dental resin.

In a second aspect of the invention, the metformin-containing dental material is a metformin-containing dental adhesive or metformin-containing dental cement comprising (i) metformin in an amount of about 0.01% to about 40% of the mass of the adhesive or cement, (ii) one or more resins in a combined amount of about 20% to about 99.99% of the mass of the adhesive or cement, and (iii) one or more curing agents in a combined amount of about 0.05% to about 5% of the mass of the adhesive or cement.

In a third aspect of the invention, the metformin-containing dental material is a metformin-containing dental composite comprising (i) metformin in an amount of about 0.01% to about 40% of the mass of the dental composite, (ii) one or more resins in a combined amount of about 5% to about 60% of the mass of the dental composite, and (iii) one or more fillers in a combined amount of about 30% to about 90% of the mass of the composite.

In each aspect of the invention, the one or more resins that comprise the metformin-containing dental materials of the invention may be, but are not limited to, bisphenol glycidyl methacrylate (bis-GMA), triethylene glycol dimethacrylate (TEGDMA), 2-hydroxyethyl methacrylate (HEMA), urethane dimethacrylate (UDMA), pyromellitic acid glycerol dimethacrylate (PMGDM), ethoxylated bisphenol A dimethacrylate (EBPADMA), Bis[2-(methacryloyloxy)ethyl] phosphate (BisMEP), methacryloyloxyethyl phthalate (MEP), methacrylate-modified polyalkenoic acid, pyromellitic dimethacrylate (PMDM), glycerol dimethacrylate/maleate adduct, glycerol dimethacrylate/succinate adduct, 2-acetoacetoxyethyl methacrylate, methacryloyloxyethyl maleate, a hydrophobic monomer, a hydrophilic monomer, a poly acid-modified polymer, a low shrinkage resin, a non-shrinkage resin, a light-cured polymer, a self-cured polymer, a duel cured polymer, and a heat-cured polymer.

In each aspect of the invention, the curing agents that comprise the metformin-containing dental materials of the invention comprise one or more of a photo-curing agent and a chemical curing agent. Suitable photo-curing agents include, but are not limited to camphorquinone (CQ), ethyl 4-N,N-dimethylaminobenzoate and phenylbis (2,4,6-triemthylbenzoyl) phosphine oxide. Suitable chemical curing agents include, but are not limited to, benzoyl peroxide (BPO).

In each aspect of the invention, the fillers that comprise the metformin-containing dental materials of the invention comprise one or more of a glass filler, a ceramic filler, and a polymer-based filler. Suitable glass fillers include, but are not limited to, barium boroaluminosilicate glass particles, fluoroaluminosilicate glass particles, fluoroaluminosilicate glass particles modified with a polyalkenoic acid, strontium-alumino-fluoro-silicate glass particles, silicon dioxide particles, fluoroaluminosilicate glass particles, fluoroaluminosilicate glass particles modified with a polycarboxylic acid, ytterbium tri-fluoride particles, and fiber glass particles. In certain aspects, the filler is barium boroaluminosilicate. In aspects where the filler is a ceramic filler, suitable ceramic fillers include, but are not limited to, a porcelain filler, a quartz filler, and a zirconia filler.

The metformin-containing dental materials defined in each of the aspects of the invention may also comprise one or more antibacterial agents. Acceptable antibacterial agents include, but not limited to, antibacterial monomers, quaternary ammonium salts (QASs), quaternary ammonium monomers (QAMs), silver-containing nanoparticles (NanoAgs), chlorhexidine particles, TiO₂ particles, and ZnO particles. The combined amount of the antibacterial agents included in the dental material is about 0.5% to about 50% of the mass of the dental material in which they are included.

When antibacterial monomers are included in the dental materials, the antibacterial monomers include, but are not limited to, dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). The combined amount of the antibacterial monomers included in the dental material is about 0.5% to about 50% of the mass of the dental material in which they are included.

When present, the amount of quaternary ammonium salts included in the dental material is about 0.01% to about 30% of the mass of the dental material in which they are included, preferably about 2% to about 15% of the mass of the dental material in which they are included.

When present, the amount of silver-containing nanoparticles included in the dental material is about 0.01% to about 20% of the mass of the dental material in which they are included, preferably 0.08% to 10% of the mass of the dental material in which they are included.

The metformin-containing dental materials defined in each of the aspects of the invention may also comprise one or more remineralization agents. Suitable remineralization agents include, but are not limited to, nanoparticles of amorphous calcium phosphate (NACP). The NACP particles may range in size from about 10 nm to about 500 nm. In certain aspects, the NACP particles range in size from about 50 nm to about 300 nm, about 50 nm to about 200 nm, or about 75 nm to about 200 nm. When NACP is present, it may make up between about 1% and 80% of the mass of the dental material in which it is included.

The metformin-containing dental materials defined in each of the aspects of the invention may also comprise one or more of acidic methacrylate or acrylate-based monomers. The combined amount of the acidic methacrylate or acrylate-based monomers included in the dental material is about 1% to about 50% of the mass of the dental material in which they are included.

The metformin-containing dental materials defined in each of the aspects of the invention may also comprise one or more protein-repellant materials. Acceptable protein-repellant materials include, but are not limited to, 2-methacryloyloxyethyl phosphorylcholine (MPC), poly(hydroxyethyl methacrylate) (HEMA) and derivatives thereof, and poly(N-isopropylacrylamide) and derivatives thereof. The combined amount of the protein repellant materials included in the dental material is about 0.5% to about 50% of the mass of the dental material in which they are included.

The metformin-containing dental materials defined in each of the aspects of the invention may be used in a wide range of different dental applications and procedures including, but not limited to, inside a tooth cavity, under a tooth cavity restoration, on a tooth surface, on a tooth root surface, in a periodontal pocket, or in a root canal.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment or aspect disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF THE DRAWINGS

FIG. 1. Evaluation of cell proliferation of dental pulp stem cells (DPSCs) attaching to dental resins (mean±sd; n=6). The cell amount increased with time due to proliferation. Control group and metformin resin group had similar cell proliferation (p>0.1), indicating that metformin did not adversely affect cell attachment and proliferation.

FIG. 2. Representative live/dead cell fluorescence staining images of DPSCs on resins with or without metformin. There was no noticeable difference between the two groups.

FIG. 3. qPCR results for odontoblast differentiation of DPSCs on resins with or without metformin (mean±sd; n=6). The gene expressions of dentin sialophosphoprotein (DSPP) (A), dentin matrix phosphoprotein 1 (DMP-1) (B), alkaline phosphatase (ALP) (C) and runt-related transcription factor 2 (Runx2) (D) were analyzed at 1, 7 and 14 days. Compared to control resin (left column in each pair), there was significant up-regulation of odontoblast-related genes: DSPP (A) at day 7 and day 14, and DMP-1(B) at day 1 and day 7. Mineral-related gene expression of ALP (C) at 7 and 14 days were also higher than that of DPSCs on control resin (p<0.05).

FIG. 4. Alkaline phosphatase (ALP) activity of DPSCs on resins with or without metformin (mean±sd; n=6). Greater ALP activity values in metformin group were obtained than those of control group (left column in each pair) at day 14 and day 21 (p<0.05).

FIGS. 5A-5B. Alizarin red staining (ARS) of mineral synthesis by DPSCs on resins with or without metformin. (A) Representative ARS staining images of DPSC-seeded resin disks. (B) DPSC mineral synthesis quantification (mean±sd; n=6). The amount of mineral synthesis by DPSCs on metformin resin was greatly increased with culture time, and at day 14 and day 21 was much more than that on control resin (left column in each pair) (p<0.05).

### DETAILED DESCRIPTION

The objectives of the present invention included the development of metformin (1,1-dimethylbiguanide hydrochloride)-containing materials for dental applications, and investigations into the effects of metformin incorporation on DPSC viability, proliferation and odontoblastic differentiation. It was hypothesized that: (1) metformin-containing dental resins would not adversely affect DPSC attachment and viability compared to resins lacking metformin; (2) metformin-containing dental resins would substantially enhance the odontogenic gene expression and alkaline phosphatase activity of DPSCs growing on the resins, compared to control resin without metformin; (3) DPSCs on metformin-containing dental resins would synthesize much more mineral than that on the resin without metformin. As described herein, these objectives were fully realized.

As summarized above, the present invention is directed to metformin-containing dental materials for use in dental products and applications. The metformin-containing dental materials of the invention are those that comprise (i) metformin in an amount of about 0.01% to about 40% of the mass of the dental material and (ii) one or more resins in a combined amount of about 20% to about 99.99% of the mass of the dental material.

The metformin-containing dental materials of the invention include, but are not limited to, metformin-containing dental primers, metformin-containing dental resins, metformin-containing dental bases, metformin-containing dental liners, metformin-containing tooth carie restorations, metformin-containing dental composites (such as flowable composites, low-shrinkage composites, and non-shrinking composites), metformin-containing bonding agents, metformin-containing adhesives, metformin-containing sealants (such as pit and fissure sealants), metformin-containing varnish, metformin-containing cements (such as orthodontic cements, crown cements, and inlay/onlay cements), metformin-containing coatings (such as tooth coatings and root surface coatings), and other metformin-containing dental products.

The metformin-containing dental materials of the invention are further defined in the following descriptions of non-limiting aspects and examples of the invention.

### Metformin-Containing Dental Resins

In a first aspect, the metformin-containing dental material is a metformin-containing dental resin. The metformin-containing dental resins comprise (i) metformin and (ii) one or more resins. The dental resins of the invention may include additional components, as disclosed herein. The amount of metformin present in the dental resins of the invention can vary depending on the identity of the resins and other components, if any, that may be included in the dental resin. However, the amount of metformin present in the dental resins of the invention will generally range from about 0.01% to about 40% of the mass of the dental resin. In some aspects, the amount of metformin will range from about 1% to about 35%, from about 5% to about 30%, from about 10% to about 30%, or from about 15% to about 25% of the mass of the dental resin. In some other aspects, the amount of metformin will be at least about 0.01%, 0.1%, 1%, 5%, 10%, 15%, 20%, 25%, 30%, 35%, or 40% of the mass of the dental resin. In further other aspects, the amount of metformin is about 0.01%, 0.1%, 1%, 5%, 10%, 15%, 20%, 25%, 30%, 35%, or 40% of the mass of the dental resin.

Similarly, the combined amount of the one or more resins present in the dental resins of the invention can vary depending on the identity of the resins and other components, if any, that may be included in the dental resin. However, the combined amount of the one or more resins present in the dental resins of the invention will generally range from about 20% to about 99.9% of the mass of the dental resin. In some aspects, the combined amount of the one or more resins will range from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, or from about 60% to about 70% of the mass of the dental resin. In some other aspects, the combined amount of the one or more resins will be at least about 20%, 25%, 30%, 35%, 40%, 45%, 50%, 55%, 60%, 65%, 70%, 75%, 80%, 85%, 90%, 95% or more of the mass of the dental resin. In further aspects, the combined amount of the one or more resins will be about 20%, 25%, 30%, 35%, 40%, 45%, 50%, 55%, 60%, 65%, 70%, 75%, 80%, 85%, 90% or 95% of the mass of the dental resin.

### Metformin-Containing Dental Adhesives

In a second aspect, the metformin-containing dental material is a metformin-containing dental adhesive. The metformin-containing dental adhesives comprise (i) metformin, (ii) one or more resins and (iii) one or more curing agents. The dental adhesives of the invention may include additional components, as disclosed herein. The amount of metformin present in the dental adhesives of the invention can vary depending on the identity of the resins, curing agents and other components, if any, that may be included in the dental adhesive. However, the amount of metformin present in the dental adhesives of the invention will generally range from about 0.01% to about 40% of the mass of the dental adhesive. In some aspects, the amount of metformin will range from about 1% to about 35%, from about 5% to about 30%, from about 10% to about 30%, or from about 15% to about 25% of the mass of the dental adhesive. In some other aspects, the amount of metformin will be at least about 0.01%, 0.1%, 1%, 5%, 10%, 15%, 20%, 25%, 30%, 35%, or 40% of the mass of the dental adhesive. In further other aspects, the amount of metformin is about 0.01%, 0.1%, 1%, 5%, 10%, 15%, 20%, 25%, 30%, 35%, or 40% of the mass of the dental adhesive.

Similarly, the combined amount of the one or more resins present in the dental adhesives of the invention can vary depending on the identity of the resins, curing agents and other components, if any, that may be included in the dental adhesive. However, the combined amount of the one or more resins present in the dental adhesives of the invention will generally range from about 20% to about 99.9% of the mass of the dental adhesives. In some aspects, the combined amount of the one or more resins will range from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, or from about 60% to about 70% of the mass of the dental adhesives. In some other aspects, the combined amount of the one or more resins is at least about 20%, 25%, 30%, 35%, 40%, 45%, 50%, 55%, 60%, 65%, 70%, 75%, 80%, 85%, 90%, 95% or more of the mass of the dental adhesives. In further aspects, the combined amount of the one or more resins is about 20%, 25%, 30%, 35%, 40%, 45%, 50%, 55%, 60%, 65%, 70%, 75%, 80%, 85%, 90% or 95% of the mass of the dental adhesives.

The combined amount of the one or more curing agents present in the dental adhesives of the invention can vary depending on the identity of the resins, curing agents and other components, if any, that may be included in the dental adhesive. However, the combined amount of the one or more curing agents present in the dental adhesives of the invention will generally range from about 0.5% to about 5% of the mass of the dental adhesive. In some aspects, the combined amount of the one or more curing agents will range from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 0.5% to about 1%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 2% to about 4.5%, from about 2% to about 4%, from about 2% to about 3.5%, from about 2% to about 3%, from about 2% to about 2.5%, from about 2.5% to about 4.5%, from about 2.5% to about 4%, from about 2.5% to about 3.5%, from about 2.5% to about 3%, from about 3% to about 4.5%, from about 3% to about 4%, from about 3% to about 3.5%, from about 3.5% to about 4.5%, from about 3.5% to about 4%, or from about 45% to about 4.5% of the mass of the dental adhesive. In some other aspects, the combined amount of the one or more curing agents will be at least about 0.5%, 1%, 1.5%, 2%, 2.5%, 3%, 3.5%, 4%, 4.5%, 5% or more of the mass of the dental adhesive. In further aspects, the combined amount of the one or more curing agents is about 0.5%, 1%, 1.5%, 2%, 2.5%, 3%, 3.5%, 4%, 4.5%, or 5% of the mass of the dental adhesive.

### Metformin-Containing Dental Cements

In a related aspect, the metformin-containing dental material is a metformin-containing dental cement. The metformin-containing dental cements comprise (i) metformin, (ii) one or more resins and (iii) one or more curing agents. The dental cements of the invention may include additional components, as disclosed herein. The amount of metformin present in the dental cements of the invention can vary depending on the identity of the resins, curing agents and other components, if any, that may be included in the dental cements. However, the amount of metformin present in the dental cements of the invention will generally range from about 0.01% to about 40% of the mass of the dental cement. In some aspects, the amount of metformin will range from about 1% to about 35%, from about 5% to about 30%, from about 10% to about 30%, or from about 15% to about 25% of the mass of the dental cement. In some other aspects, the amount of metformin will be at least about 0.01%, 0.1%, 1%, 5%, 10%, 15%, 20%, 25%, 30%, 35%, or 40% of the mass of the dental cement. In further other aspects, the amount of metformin is about 0.01%, 0.1%, 1%, 5%, 10%, 15%, 20%, 25%, 30%, 35%, or 40% of the mass of the dental cement.

Similarly, the combined amount of the one or more resins present in the dental cements of the invention can vary depending on the identity of the resins, curing agents and other components, if any, that may be included in the dental cement. However, the combined amount of the one or more resins present in the dental cements of the invention will generally range from about 20% to about 99.9% of the mass of the dental cement. In some aspects, the combined amount of the one or more resins will range from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, or from about 60% to about 70% of the mass of the dental cement. In some other aspects, the combined amount of the one or more resins is at least about 20%, 25%, 30%, 35%, 40%, 45%, 50%, 55%, 60%, 65%, 70%, 75%, 80%, 85%, 90%, 95% or more of the mass of the dental cement. In further aspects, the combined amount of the one or more resins is about 20%, 25%, 30%, 35%, 40%, 45%, 50%, 55%, 60%, 65%, 70%, 75%, 80%, 85%, 90% or 95% of the mass of the dental cement.

The combined amount of the one or more curing agents present in the dental cements of the invention can vary depending on the identity of the resins, curing agents and other components, if any, that may be included in the dental cement. However, the combined amount of the one or more curing agents present in the dental cements of the invention will generally range from about 0.5% to about 5% of the mass of the dental cement. In some aspects, the combined amount of the one or more curing agents will range from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 0.5% to about 1%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 2% to about 4.5%, from about 2% to about 4%, from about 2% to about 3.5%, from about 2% to about 3%, from about 2% to about 2.5%, from about 2.5% to about 4.5%, from about 2.5% to about 4%, from about 2.5% to about 3.5%, from about 2.5% to about 3%, from about 3% to about 4.5%, from about 3% to about 4%, from about 3% to about 3.5%, from about 3.5% to about 4.5%, from about 3.5% to about 4%, or from about 45% to about 4.5% of the mass of the dental cement. In some other aspects, the combined amount of the one or more curing agents is at least about 0.5%, 1%, 1.5%, 2%, 2.5%, 3%, 3.5%, 4%, 4.5%, 5% or more of the mass of the dental cement. In further aspects, the combined amount of the one or more curing agents is about 0.5%, 1%, 1.5%, 2%, 2.5%, 3%, 3.5%, 4%, 4.5%, or 5% of the mass of the dental cement.

Metformin-containing Dental Composites

In a third aspect, the metformin-containing dental material is a metformin-containing dental composite. The metformin-containing dental composites comprise (i) metformin, (ii) one or more resins and (iii) one or more fillers. The dental composites of the invention may include additional components, as disclosed herein. The amount of metformin present in the dental composites of the invention can vary depending on the identity of the resins, fillers and other components, if any, that may be included in the dental composites. However, the amount of metformin present in the dental composites of the invention will generally range from about 0.01% to about 40% of the mass of the dental composite. In some aspects, the amount of metformin will range from about 1% to about 35%, from about 5% to about 30%, from about 10% to about 30%, or from about 15% to about 25% of the mass of the dental composite. In some other aspects, the amount of metformin will be at least about 0.01%, 0.1%, 1%, 5%, 10%, 15%, 20%, 25%, 30%, 35%, or 40% of the mass of the dental composite. In further other aspects, the amount of metformin is about 0.01%, 0.1%, 1%, 5%, 10%, 15%, 20%, 25%, 30%, 35%, or 40% of the mass of the dental composite.

Similarly, the combined amount of the one or more resins present in the dental composites of the invention can vary depending on the identity of the resins, fillers and other components, if any, that may be included in the dental composite. However, the combined amount of the one or more resins present in the dental composites of the invention will generally range from about 5% to about 60% of the mass of the dental composite. In some aspects, the combined amount of the one or more resins will range from about 5% to about 55%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, 10% to about 55%, from about 10% to about 50%, from about 10% to about 45%, from about 10% to about 40%, from about 10% to about 35%, from about 10% to about 30%, from about 10% to about 25%, from about 10% to about 20%, from about 10% to about 15%, 15% to about 55%, from about 15% to about 50%, from about 15% to about 45%, from about 15% to about 40%, from about 15% to about 35%, from about 15% to about 30%, from about 15% to about 25%, from about 15% to about 20%, 20% to about 55%, from about 20% to about 50%, from about 20% to about 45%, from about 20% to about 40%, from about 20% to about 35%, from about 20% to about 30%, from about 20% to about 25%, 25% to about 55%, from about 25% to about 50%, from about 25% to about 45%, from about 25% to about 40%, from about 25% to about 35%, from about 25% to about 30%, 30% to about 55%, from about 30% to about 50%, from about 30% to about 45%, from about 30% to about 40%, from about 30% to about 35%, 35% to about 55%, from about 35% to about 50%, from about 35% to about 45%, from about 35% to about 40%, 40% to about 55%, from about 40% to about 50%, from about 40% to about 45%, 45% to about 55%, from about 45% to about 50%, or from about 50% to about 60% of the mass of the dental composite. In some other aspects, the combined amount of the one or more resins is at least about 5%, 10%, 15%, 20%, 25%, 30%, 35%, 40%, 45%, 50%, 55%, 60% or more of the mass of the dental composite. In further aspects, the combined amount of the one or more resins is about 5%, 10%, 15%, 20%, 25%, 30%, 35%, 40%, 45%, 50%, 55%, or 60% of the mass of the dental composite.

The combined amount of the one or more fillers present in the dental composites of the invention can vary depending on the identity of the resins, fillers and other components, if any, that may be included in the dental composite. However, the combined amount of the one or more fillers present in the dental composites of the invention will generally range from about 30% to about 90% of the mass of the dental composite. In some aspects, the combined amount of the one or more fillers will range from about 30% to about 85%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, or from about 60% to about 70% of the mass of the dental composite. In some other aspects, the combined amount of the one or more fillers is at least about 30%, 35%, 40%, 45%, 50%, 55%, 60%, 65%, 70%, 75%, 80%, 85%, 90% more of the mass of the dental composite. In further aspects, the combined amount of the one or more fillers is about 30%, 35%, 40%, 45%, 50%, 55%, 60%, 65%, 70%, 75%, 80%, 85%, or 90% of the mass of the dental composite.

### Resins

The one or more resins that may be used in the dental materials of the invention (e.g. the metformin-containing dental resins, metformin-containing dental adhesives, metformin-containing dental cements, and metformin-containing dental composites) are one or more of bisphenol glycidyl methacrylate (bis-GMA), triethylene glycol dimethacrylate (TEGDMA), 2-hydroxyethyl methacrylate (HEMA), urethane dimethacrylate (UDMA), pyromellitic acid glycerol dimethacrylate (PMGDM), ethoxylated bisphenol A dimethacrylate (EBPADMA), Bis[2-(methacryloyloxy)ethyl] phosphate (BisMEP), methacryloyloxyethyl phthalate (MEP), methacrylate-modified polyalkenoic acid, pyromellitic dimethacrylate (PMDM), glycerol dimethacrylate/maleate adduct, glycerol dimethacrylate/succinate adduct, 2-acetoacetoxyethyl methacrylate, methacryloyloxyethyl maleate, a hydrophobic monomer, a hydrophilic monomer, a poly acid-modified polymer, a low shrinkage resin, a non-shrinkage resin, a light-cured polymer, a self-cured polymer, a duel cured polymer, and a heat-cured polymer. The one or more resins

The dental materials of the invention may comprise 1, 2, 3, 4, 5, 6, 7 or more of the resins provided herein. In one non-limiting example, the one or more resins used in the dental materials comprise each of PMGDM, EBPADMA, HEMA, and Bis-GMA. In a specific example, the one or more resins used in the dental materials comprise 44.5% PMGDM, 39.5% EBPADMA, 10% HEMA, and 5% Bis-GMA, in association with 1% phenylbis (2,4,6-triemthylbenzoyl) phosphine oxide (BAPO) as a curing agent. Alternatively, the one or more resins used in the dental materials comprise BiS-GMA and TEGDMA. Further, the one or more resins used in the dental materials comprise PMGDM and EBPADMA.

### Curing Agents

The one or more curing agents that may be used in the dental materials of the invention (e.g. the metformin-containing dental resins, metformin-containing dental adhesives, metformin-containing dental cements, and metformin-containing dental composites) are one or more of a photo-curing agent and a chemical curing agent. For example, camphorquinone (CQ), ethyl 4-N,N-dimethylaminobenzoate, phenylbis (2,4,6-triemthylbenzoyl) phosphine oxide, or combinations thereof may be included in the dental materials, rendering the resins light-curable. In another example, benzoyl peroxide (BPO) may be included in the dental materials, rendering the resins chemically-curable. In yet another example, one or more light-curable compounds and one or more chemical-curable compounds may be included in the dental materials. In a particular example, about 0.2% CQ and about 0.8% ethyl 4-N,N-dimethylaminobenzoate may be included in the dental materials to render the resulting material light-curable. In another example, 1% phenylbis (2,4,6-triemthylbenzoyl) phosphine oxide (BAPO) may be included in the dental materials to render the resulting material light-curable. In further example, CQ and BPO may be included in the dental materials to render the resulting material light-curable and chemically-curable.

### Fillers

The one or more fillers that may be used in the dental materials of the invention (e.g. the metformin-containing dental resins, metformin-containing dental adhesives, metformin-containing dental cements, and metformin-containing dental composites) are one or more of a glass filler, a ceramic filler, and a polymer-based filler. Suitable glass fillers include, but are not limited to, barium boroaluminosilicate glass particles, fluoroaluminosilicate glass particles, fluoroaluminosilicate glass particles modified with a polyalkenoic acid, strontium-alumino-fluoro-silicate glass particles, silicon dioxide particles, fluoroaluminosilicate glass particles, fluoroaluminosilicate glass particles modified with a polycarboxylic acid, ytterbium tri-fluoride particles, and a fiber glass particles. In certain aspects, the filler is barium boroaluminosilicate. In aspects where the filler is a ceramic filler, suitable ceramic fillers include, but are not limited to, a porcelain filler, a quartz filler, and a zirconia filler.

The fillers used in the dental materials may be a silanized filler. Fillers can be silanized using 4% 3-methacryloxypropyltrimethoxysilane and 2% n-propylamine.

In one non-limiting example, the one or more fillers used in the dental materials is barium boroaluminosilicate glass particles with a median size of 1.4 μm that are silanized with 4% 3-methacryloxypropyltrimethoxysilane and 2% n-propylamine.

### Antibacterial Agents

The metformin-containing dental materials defined in each of the aspects of the invention may also comprise one or more antibacterial agent. Acceptable antibacterial agents include, but not limited to, antibacterial monomers, quaternary ammonium salts (QASs), quaternary ammonium monomers (QAMs), silver-containing nanoparticles (NanoAgs), chlorhexidine particles, TiO₂ particles, and ZnO particles.

When antibacterial monomers are included in the dental materials, the antibacterial monomers include, but are not limited to, dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

When present, the amount of antibacterial monomers in the dental material is a combined amount of antibacterial monomers of from about 0.5% to about 50% of the mass of the dental material. In certain aspects, the combined amount of the antibacterial monomers is from about 1% to about 25%, from about 2.5% to about 25%, from about 2.5% to about 20%, from about 2.5% to about 15%, from about 5% to about 25%, from about 5% to about 20%, from about 5% to about 15%, from about 7.5% to about 25%, from about 7.5% to about 20%, about 7.5% to about 15%, or from about 7.5% to about 12.5% of the mass of the rechargeable dental materials.

Suitable QASs include both polymerizable monomers and non-polymerizable small molecules, and include, but are not limited to, bis(2-methacryloyloxy-ethyl) dimethyl-ammonium bromide (QADM), methacryloyloxydodecylpyridinium bromide, methacryloxylethyl benzyl dimethyl ammonium chloride, methacryloxylethyl m-chloro benzyl dimethyl ammonium chloride, methacryloxylethyl cetyl dimethyl ammonium chloride, cetylpyridinium chloride, and methacryloxylethyl cetyl ammonium chloride, QAS chlorides, QAS bromides, QAS monomethacrylates, QAS dimethacrylates, and pre-fabricated QAS particles. See U.S. Pat. No. 8,889,196, which is incorporated by reference herein in its entirety. When present, the QAS may make up between about 0.01% and about 30% of the mass of the dental material. In certain aspects, the QAS will make up between about 2% and about 25%, about 2% and about 15%, about 5% and about 20%, or about 7.5% and about 15% of the mass of the dental material, or about 1%, 2.5%, 5%, 7.5%, 10%, 12.5, 15%, 17.5%, 20%, 22.5%, 25%, 27.5% or 30% of the mass of the dental material.

Suitable NanoAgs include, but are not limited to, silver 2-ethylhexanoate salt, silver-containing glass particles and silver benzoate. In addition to silver salts, pre-formed silver nanoparticles can be used. When present, NanoAgs may make up between about 0.01% and about 20% of the mass of the dental material. In certain aspects, NanoAgs will make up between about 0.05% and about 5%, or 0.08% and about 10%, of the mass of the dental material, or about 0.01%, 0.08%, 0.15%, 0.20%, 0.25%, 0.30%, 0.35%, 0.40%, 0.45%, 0.50%, 1.0%, 1.5%, 2.0%, 2.5%, 3.0%, 3.5%, 4.0%, 4.5% or 5.0% of the mass of the dental material. In one aspect, NanoAgs makes up about 0.08% of the mass of the dental material. The silver particle size can range from about 1 nm to about 1000 nm, and in one aspect, from about 2 nm to about 500 nm.

### Remineralization Agents

The metformin-containing dental materials defined in each of the aspects of the invention may also comprise one or more remineralization agents. Suitable remineralization agents include, but are not limited to, nanoparticles of amorphous calcium phosphate (NACP). NACP comprises nanometer-sized amorphous calcium phosphate (Ca₃[PO₄]₂) particles. The use of NACP results in dental materials with high Ca and PO₄ release, excellent mechanical properties, and antibacterial properties. Dental materials that include NACP exhibit greatly increased ion release at acidic, cariogenic pH, when these ions are most needed to inhibit caries.

The NACP may make up between about 1% and about 90% of the mass of the dental materials. In certain aspects, the NACP is from about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, or about 30% to about 40% of the mass of the dental material.

The NACP particles may range in size from about 10 nm to about 1000 nm. In certain aspects, the NACP particles range in size from about 10 nm to about 500 nm, about 50 nm to about 750 nm, about 50 nm to about 500 nm, about 75 nm to about 700 nm, about 75 nm to about 3000 nm, about 100 nm to about 200 nm, or about 125 nm to about 200 nm. In other aspects, the size of the NACP particles averages about 75, 80, 85, 90, 95, 100, 105, 110, 115, 120, 125, 130, 135, 140, 145, 150, 155, 160, 165, 170, 175, 180, 185, 190, 195 or 200 nm. In further aspects, the size of the NACP particles is about 160 nm to about 170 nm. In other aspects, the size of the NACP particles average 116 nm. In yet another aspect, the size of the NACP particles average 166 nm.

The NACP particles have a relatively high specific surface area. The NACP particles have a specific surface area of about 2-206 m²/g. In certain aspects, the NACP particles have a specific surface area of about 3-150 m²/g, 4-100 m²/g, 5-75 m²/g, 6-50 m²/g, or 10-25 m²/g. In a certain aspect, the NACP particles have a specific surface area of about 15-20 m²/g. In a further aspect, the NACP particles have a specific surface area of about 17.75 m²/g.

### Acidic Methacrylate or Acrylate-Based Monomers

The metformin-containing dental materials defined in each of the aspects of the invention may also comprise one or more of acidic methacrylate or acrylate-based monomers. These monomers can be divided by their corresponding acidic functional groups: (1) carboxylic acid, (2) phosphonic acid, and (3) sulfonic acid. Monomers from each of these groups may be used to recharge calcium and phosphate ions in the same manner that PMGDM (carboxylic acid functionality) and MEP (phosphonic acid functionality) have been shown to recharge.

Suitable carboxylic acid-based monomers include, but are not limited to, pyromellitic dimethacrylate (PMDM) [25], methacryloyloxyethyl phthalate [25], methacryloyloxyethyl maleate [26], 2-hydroxyethyl methacrylate/succinate [19], glycerol dimethacrylate/maleate adduct [24], glycerol dimethacrylate/sucinate adduct [24], mono-2-(methacryloyloxy)ethyl maleate (CAM) [22], 4-methacryloyloxyethyl trimellitic acid (4-MET) [23], 10-methacryloyloxydecyl malonic acid (MAC-10) [23], N-methacryloyl-1-aminosalicylic acid (MASA) [23], N-methacryloyl glycine (NMGLY) [23], biphenyl dimethacrylate or 4,4′-dimethacryloyloxyethyloxycarbonylbiphenyl-3,3′-dicarboxylic acid (BPDM) [25], butan-1,2,3,4-tetracarboxylic acid di-2-hydroxyethylmethacrylate ester (TCB) [25], ortho-(N-methacryloyl amino) benzoic acid (o-MABA) [21], meta-(N-methacryloyl amino) benzoic acid (m-MABA) [21], para-(N-methacryloyl amino) benzoic acid (p-MABA) [21], 2-(N-methacryloyl amino) terephthalic acid (2-MATPA) [21], 5-(N-methacryloyl amino) isophthalic acid (5-MAIPA) [21], and 4-methacryloxy phthalic acid (4-MPA) [21].

Suitable phosphonic acid-based monomers include, but are not limited to, 2-hydroxyethyl methacrylate phosphate [25], 10-Methacryloyldecyl Dihydrogen Phosphate (MDP) [25], 1,3-glycerol dimethacrylate phosphate (PAM) [22], Glycerol dimethacrylate ester of phosphoric acid (GDMP) [23], methacryloyloxyethyl phenyl hydrogen phosphate (MEP-P) [23], methacryloyloxypropyl dihydrogen phosphate (MPP) [23], dipentaerythrolpentaacryloyl dihydrogen phosphate (PENTA-P) [23], vinylphosphonic acid (VPA) [23], 4-vinylbenzylphosphonic acid (VBPA) [23], ethyl 2-[4-(dihydroxyphosphoryl)-2-oxabutyl]acrylate (EAEPA) [23], (2,4,6-trimethylphenyl 2-[4-(dihydroxyphosphoryl)-2-oxabutyl]acrylate (MAEPA) [23], 2-[4-(dihydroxyphosphoryl)-2-oxabutyl]acrylonitrile (NAEPA) [23], and 2-[4-(dihydroxyphosphoryl)-2-oxabutyl] acrylic acid (CAEPA) [23].

Suitable sulfonic acid-based monomers include, but are not limited to, 2-acrylamido-2-methylpropane sulfonic acid [23], and 2-sulfoethyl methacrylate [20].

The combined amount of the one or more acidic methacrylate and acrylate-based monomers in the dental materials is about 1% to about 50% by mass of the dental material. In certain aspects, the combined amount of the one or more acidic methacrylate and acrylate-based monomers in the dental material is from about 1% to about 40%, from about 2.5% to about 50%, from about 2.5% to about 45%, from about 2.5% to about 40%, from about 2.5% to about 35%, from about 2.5% to about 30%, from about 2.5% to about 25%, from about 2.5% to about 20%, from about 2.5% to about 15%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 7.5% to about 50%, from about 7.5% to about 45%, from about 7.5% to about 40%, from about 7.5% to about 35%, from about 7.5% to about 30%, from about 7.5% to about 25%, from about 7.5% to about 20%, about 7.5% to about 15%, from about 7.5% to about 12.5%, from about 10% to about 50%, from about 10% to about 45%, from about 10% to about 40%, from about 10% to about 35%, from about 10% to about 30%, from about 10% to about 25%, from about 10% to about 20%, or about 40% to 50% of the mass of the dental materials.

### Protein-Repellant Materials

The metformin-containing dental materials defined in each of the aspects of the invention may also comprise one or more protein repellant materials. The protein-repellent agents inhibit adsorption of bacteria to the dental products, thereby enhancing their anti-cariogenic properties. Acceptable protein-repellant materials include, but are not limited to, 2-methacryloyloxyethyl phosphorylcholine (MPC), poly(hydroxyethyl methacrylate) (HEMA) and derivatives thereof, and poly(N-isopropylacrylamide) and derivatives thereof. The amount of protein-repellant agent in the dental materials ranges from about 0.5% to about 50% of the mass of the dental material. In certain aspects, the range is from about 1% to about 25%, about 2.5% to about 20%, about 4% to about 15%, or about 5% to about 12.5% of the mass of the dental material. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10% of the mass of the dental material.

Dental materials encompassed within the aspects of the invention include those provided in Table 1 that are discussed in the Examples below.

TABLE 1

| | PEHB | Barium boroaluminosilicate | Metformin |
|---|---|---|---|
| | (mass %) | glass particles (mass %) | (mass %) |
| Dental Material #1 | 25 | 75 | — |
| Dental Material #2 | 25 | 55 | 20 |

PEHB referenced in Table 1 comprises each of the resins shown in Table 2, along with BAPO as a curing agent.

TABLE 2

| | PMGDM | EBPADMA | HEMA | Bis-GMA | BAPO |
|---|---|---|---|---|---|
| | (mass %) | (mass %) | (mass %) | (mass %) | (mass %) |
| PEHB | 44.5 | 39.5 | 10 | 5 | 1 |

PMGDM: pyromellitic glycerol dimethacrylate; EBPADMA: ethoxylated bisphenol A dimethacrylate; Bis-GMA: bisphenol A glycidyl dimethacrylate; HEMA: 2-hydroxyethyl methacrylate; BAPO: phenylbis (2,4,6-trimethylbenzoyl) phosphine oxide.

### Examples

### Material and Methods

### Preparation of Experimental Resin Disks

The resin consisted of 44.5% (all mass fractions unless otherwise noted) pyromellitic glycerol dimethacrylate (PMGDM; Hampford, Stratford, Conn.), 39.5% ethoxylated bisphenol A dimethacrylate (EBPADMA; Sigma-Aldrich, St. Louis, Mo.), 10% 2-hydroxyethyl methacrylate (HEMA; Esstech, Essington, Pa.), and 5% bisphenol A glycidyl dimethacrylate (BisGMA; Esstech), following a previous study [29]. This resin is referred to as PEHB. PMGDM and EBPADMA had a low cytotoxicity similar to other dental methacrylates [30]. As an acidic adhesive monomer, PMGDM is beneficial to the adhesion between dentin and composite [31]. HEMA was added to improve the co-monomer diffusion into the demineralized dentin, according to a previous study [32]. BisGMA was chosen because of its functions in improving the crosslink of monomers. 1% of phenyl bis(2,4,6-trimethylbenzoyl) phosphine oxide (BAPO; Sigma-Aldrich) was added for photopolymerization. For mechanical reinforcement, barium boroaluminosilicate glass particles (Dentsply, Milford, Del.) with a median size of 1.4 μm were silanized with 4% 3-methacryloxypropyltrimethoxysilane and 2% n-propylamine, as previously described [30]. Then metformin (1,1-dimethylbiguanide hydrochloride; Sigma-Aldrich) was incorporated into the resin. Two groups were tested:

(1) 25% PEHB resin+75% glass particles (Control group);

(2) 25% PEHB resin+55% glass particles+20% metformin (Metformin group).

The filler level of 70% yielded a cohesive paste. The samples used for cell experiments were prepared following a previous study [24]. Briefly, the cover of a sterile 96-well plate (Costar, Corning Inc., NY) was used as a mold to make resin disks approximately 8 mm in diameter and 1 mm in thickness, which were photo-cured for 1 minute. The cured resin disks were immersed in distilled water at 37° C. with stirring for 24 h to remove any unpolymerized monomers, and then sterilized by ultraviolet ray before use.

### Cells Culture

Before dental pulp stem cell (DPSC) collection, clinically healthy dental pulp tissues were obtained from human adult third molars that were removed from individuals who had their teeth extracted due to orthodontic treatment. The procedure was approved by the Institutional Review Board of the University of Maryland Baltimore. Then DPSCs were isolated and characterized as described in a previous study [33]. Briefly, pulp tissues were minced and digested in a solution of 3 mg/mL of collagenase type I and 4 mg/mL dispase for 30-60 min at 37° C. Then cell suspension was obtained by passing the digested tissue through a 70-μm cell strainer. After that, cells were pelleted and seeded into culture dishes, and incubated with Dulbecco's Modified Eagle's medium (DMEM, Gibco, Grand Island, N.Y.) supplemented with 10% fetal bovine serum (FBS, Invitrogen, Carlsbad, Calif.), 1% penicillin/streptomycin at 37° C. with 5% CO₂. After 48 h, non-adherent cells were removed and the medium was replaced every 2 days. After approximately 1-2 weeks, the culture became subconfluent, and the cells were collected by trypsinization and subcultured into new medium at 5,000 cells/cm². A previous study [34] confirmed that DPSCs generated from this method expressed surface markers characteristic of mesenchymal stem cells (MSCs) (CD29, CD44, CD166, CD73), and were negative for typical hematopoietic (CD34, CD45, CD14). The 4th passage DPSCs were used in the following experiments.

Each resin disk was placed into a well of a 48-well plate with culture medium for 2 h at 37° C. Then DPSCs were seeded with different initial concentrations in 1 mL medium in each well for the different experiments, which are described in the following sections. Then the well plates were cultured in a CO₂ incubator at 37° C.

### Cell Viability Assay

To evaluate whether mixing metformin into resin would harm the attached DPSCs, cell viability on the resin disks with or without metformin was assessed using the cell counting kit-8 (CCK-8, Endo Life Sciences, Farmingdale, N.Y.), according to the manufacturer's instructions. CCK-8 is based on the water-soluble tetrazolium salt, WST-8 reaction that could produce an orange water-soluble formazan dye in an amount that is directly related to the number of viable cells. Firstly, each well with a resin disk was seeded with 1 mL of DPSC suspension at a seeding density of 5,000 cells/well [24]. The medium was replaced every two days. Cell proliferation at 1, 4, 7 and 14 day was measured using the cell counting kit. The resin disks with DSPCs were rinsed with phosphate buffered saline (PBS) and transferred into a new 48-well plate, then 300 μL CCK-8 dye was added into each well and placed in a CO₂ incubator for 4 h. Finally, the number of viable cells was determined by measuring the absorbance of the orange colored formazan at optical density of 450 nm (OD_{450 nm}) with a microplate reader (SpectraMax M5, Molecular Devices, Sunnyvale, Calif.). Six disks were tested for each group at each time point.

### Live Dead Staining

Separate resin disks were seeded with cells and cultured for live/dead staining to examine the growth of DPSCs on resins with or without metformin, as described previously. At each time points (1, 4, 7 or 14 day), the resin disks were taken out from each well of 48-well plate, washed by PBS and immersed at 37° C. for 15 min in a live/dead staining solution (Sigma-Aldrich) containing 2 μM Calcein AM and 2 μM Propidium Iodide [33]. Then the samples were observed at an inverted fluorescence microscope (Eclipse TE-20005, Nikon, Japan) equipped with a digital camera. Three random locations of each disk were imaged, with 4 disks yielding 12 images for each group at time point.

Real Time Quantitative PCR (qPCR)

Quantitative real-time reverse transcription polymerase chain reaction (qPCR) was used to measure the gene expression of odontoblastic differentiation in DPSCs after culturing on resins with or without metformin. The total RNA were extracted using the PureLink RNA Mini Kit (Invitrogen), and then reverse-transcribed into cDNA by a High-Capacity cDNA Reverse Transcription kit (Applied Biosystems, Foster City, Calif.). The expression levels of odontogenic differentiation relative genes markers, including dentin sialophosphoprotein (DSPP), dentin matrix phosphoprotein-1 (DMP-1), alkaline phosphatase (ALP) and runt-related transcription factor 2 (Runx2), were determined by qPCR using SYBR Green PCR Master Mix (Applied Biosystems), as previously described [24]. The housekeeping gene GAPDH was used as an internal control to normalize the expression levels of different genes. The sequences of human specific primers used for the amplification of the indicated genes were synthesized commercially (Sigma-Aldrich) and are listed in Table 3. qPCR data collection and analyses were performed using an Applied Biosystems Prism 7000 Sequence Detection System, and relative expression was evaluated using the 2^{-ΔΔCt} method and normalized by the cycle threshold (Ct) values of GAPDH, according to a previous study [24]. Ct values of DPSCs on resin disks of control group at day 1 served as the calibrator (n=6).

TABLE 3
List of primer sequences used in qPCR

| Gene | Forward primer (5′ to 3′) | Reverse primer (5′ to 3′) |
|---|---|---|
| DSPP | ATGACAGTGATAGCACATCAGA | ATTGTTACCATTGCCATTACTG |
| | (SEQ ID NO: 1) | (SEQ ID NO: 2) |
| DMP-1 | CAGTGAGGAAGATGGCCA | CTTGGCAGTCATTGTCATCTT |
| | (SEQ ID NO: 3) | (SEQ ID NO: 4) |
| ALP | CGGATCCTGACCAAAAACC | TCATGATGTCCGTGGTCAAT |
| | (SEQ ID NO: 5) | (SEQ ID NO: 6) |
| Runx2 | GACTGTGGTTACCGTCATGGC | ACTTGGTTTTTCATAACAGCGGA |
| | (SEQ ID NO: 7) | (SEQ ID NO: 8) |
| GAPDH | GCACCGTCAAGGCTGAGAAC | TGGTGAAGACGCCAGTGGA |
| | (SEQ ID NO: 9) | (SEQ ID NO: 10) |

### Alkaline Phosphatase (ALP) Activity

DPSCs were seeded onto resin disks in 48-well plates at a density of 10⁴ cells/well [35]. After 24 h for the cells to attach to the resin disks, the medium was replaced by an osteogenic medium, which consisted of DMEM growth medium, 10% FBS plus 100 nM dexamethasone, 10 mM β-glycerophosphate, 0.05 mM ascorbic acid, and 10 nM 1α, 25-dihydroxyvitamin D3 (Sigma-Aldrich). At 7 and 14 days, the ALP activity of DPSCs was measured using an QuantiChrom ALP assay kit (BioAssay Systems, Cambridge, Mass.). Briefly, resin disks with DPSCs were washed by cold PBS. The attached cells were scraped and re-suspended into the lysis buffer for 30 min, then sonicated in an ice bath and centrifuged at 1,500 rpm for 5 min. Then, the ALP activity of the supernatant was measured using an ALP working solution containing 200 μL assay buffer, 5 μL Mg Acetate (final 5 mM) and 2 μL pNPP liquid substrate (10 mM), with a ratio of 20 μL sample supernatant/180 μL working solution. After mixing, the mixtures were detected shortly the absorbance at OD_{405 nm}, and again after 4 min using a microplate reader (SpectraMax M5), following the manufacturer's protocol. Finally, the ALP activity was normalized via the protein concentrations [24]. The protein concentration was quantified using a Micro BCA protein assay kit (Thermo Scientific, Rockford, Ill.), following the manufacture's protocol. Briefly, the cells lysis supernatants were mixed with the working reagent in the kit, consisting of Reagent A and Reagent B (50:1, Reagent A:B), at a volume ratio of 1:19. Then the colorimetric reaction mixtures were used for the absorbance measurements at OD_{562 nm} using a microplate reader (SpectraMax M5). Standard curves were prepared by albumin standard ampule (BSA) with different concentrations of 0, 25, 125, 250, 500, 750, 1000, 2000 μg/mL, which was used to obtain the corresponding protein concentrations.

### Alizarin Red Staining of Minerals Synthesized by the Cells

DPSCs were seeded onto resin disks in 48-well plates at a seeding density of 10⁴ cells/well [35], and cultured for 1, 7, 14 and 21 days in the osteogenic medium. Six disks were tested for each group at each time period for mineral synthesis evaluation (n=6). Then the mineral deposit by DPSCs on resin disks were examined via alizarin red staining (ARS, Millipore, Billerica, Mass.), following the manufacturer's instruction. The cells on resin disks were fixed with 1% formalin for 10 min and stained for 30 min by 2% ARS solution, which could stain calcium-rich deposits made by the cells into a dark red color [36]. Then the ARS solution was removed, the disks were washed with PBS to remove any loose alizarin red, and the disks were imaged. For quantification, the ARS-stained cells on resin disks were de-stained in 10% cetylpryidinium chloride (Sigma-Aldrich) for 15 min and the solutions were measured at OD_{652 nm} using a microplate reader (SpectraMax M5). The results were expressed by the folds of increases, with the OD value of control group on day 1 as the reference standard.

### 2.8. Statistical Analysis

The SPSS21.0 (SPSS Inc., Chicago, Ill.) software was used for the statistical analysis. Paired-sample T-test was performed to detect the significant effects of variables. All data were expressed as mean value±standard deviation (SD). The level of statistical significance was set at p<0.05.

### Results

### 3.1 Evaluation of Cell Viability

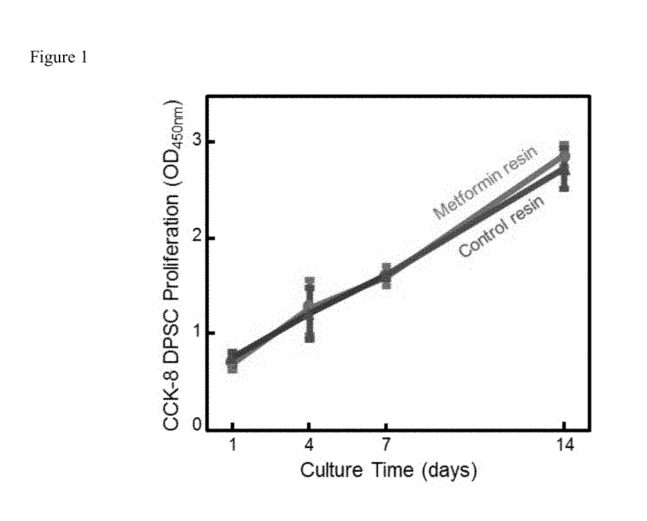

The metformin particle sizes were measured (mean±SD; n=28) to be 5.10±1.9 When metformin particles were incorporated in the resin, in order to detect any cell cytotoxicity of the metformin-containing resin, CCK-8 test was performed for DSPCs on resin at 1, 4, 7 and 14 days (mean±SD; n=6). Cell viability and proliferation were not adversely affected by the addition of metformin (FIG. 1). CCK-8 assay showed that the growth of the attached DPSCs on metformin-containing resin was similar to control without metformin (p>0.1). The number of cells on resin disks increased by approximately 4 times from 1 to 14 days; both groups had the same proliferation rate (p>0.1).

### Live/Dead Staining

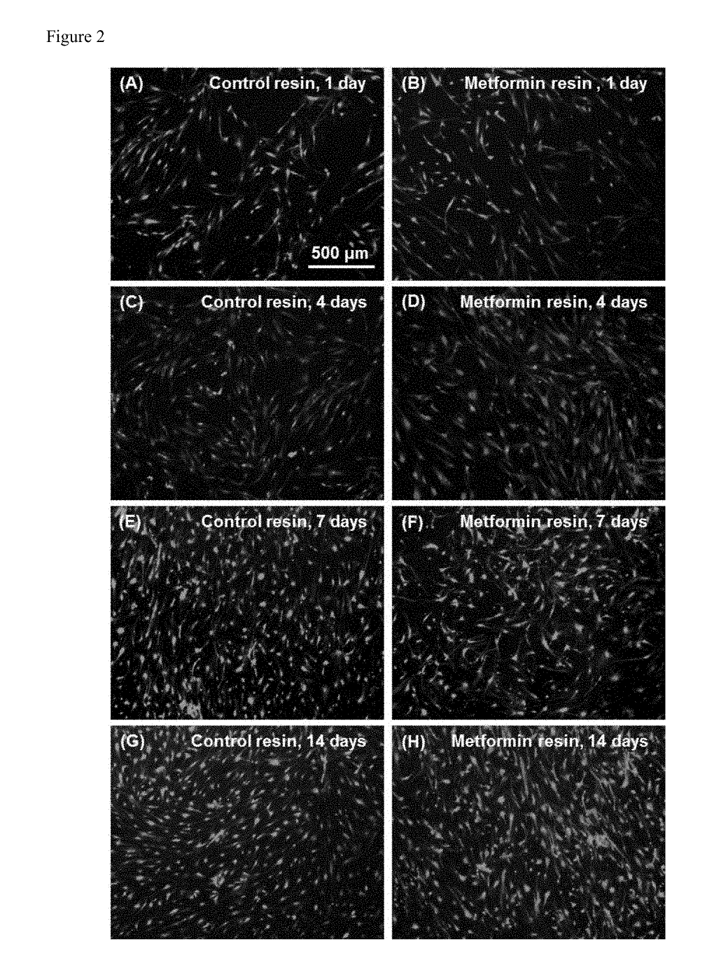

The DPSCs are relatively uniformly attached to resin in both control and metformin groups (FIG. 2). Live cells are numerous on the resin surfaces, with very few scattered red dots in the pictures. The live cell numbers increased steadily with increasing time for both groups. These results demonstrate that the resins with or without metformin both had similarly good cell compatibility, enabled cell attachment, and supported cell proliferation.

### Gene Expressions of DPSCs on Resins

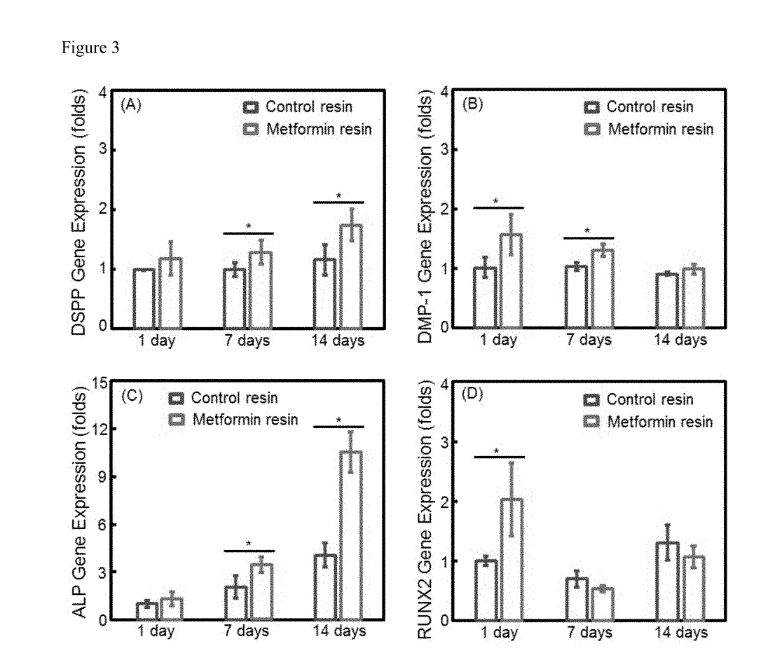

The expression of odontoblast-related genes (DSPP and DMP-1) and mineralization-related genes (ALP and Runx2) are shown in FIG. 3 (mean±SD; n=6). DSPP and ALP expressions were higher at day 7 and day 14, compared to control group (p<0.05). The expression of DMP-1 gene was significantly enhanced by the addition of metformin in resin at day 1 and day 7 (p<0.05). These qPCR results demonstrate that metformin in resin up-regulated the odontoblast- and mineralization-related genes of DPSCs on resin surfaces.

### Alkaline Phosphatase (ALP) Activity

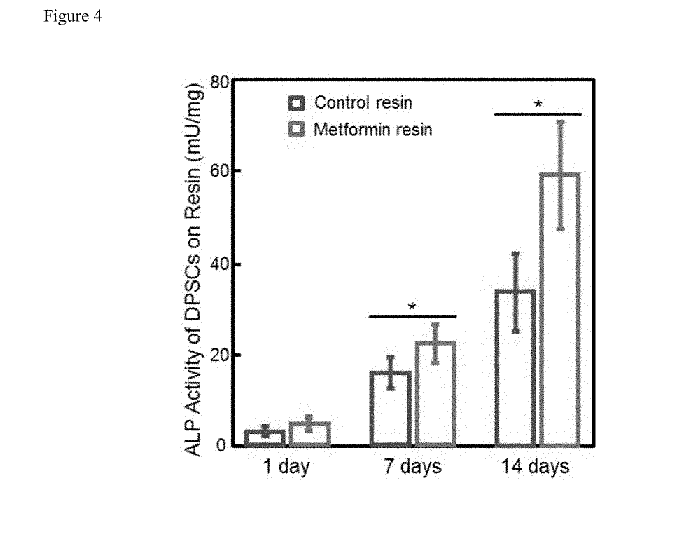

The ALP activity of DPSCs in metformin group and control group both increased with time, as the osteogenic medium was used for both groups (FIG. 4). However, remarkable differences were observed between the two groups at 7 and 14 days, with significantly higher ALP activity for metformin group than control group (p<0.05). At 14 days, the ALP activity of the metformin group was 30 folds that at 1 day (p<0.05). The ALP activity of the metformin group was and 70% higher than that of control at 14 days (p<0.05).

### Alizarin Red Staining

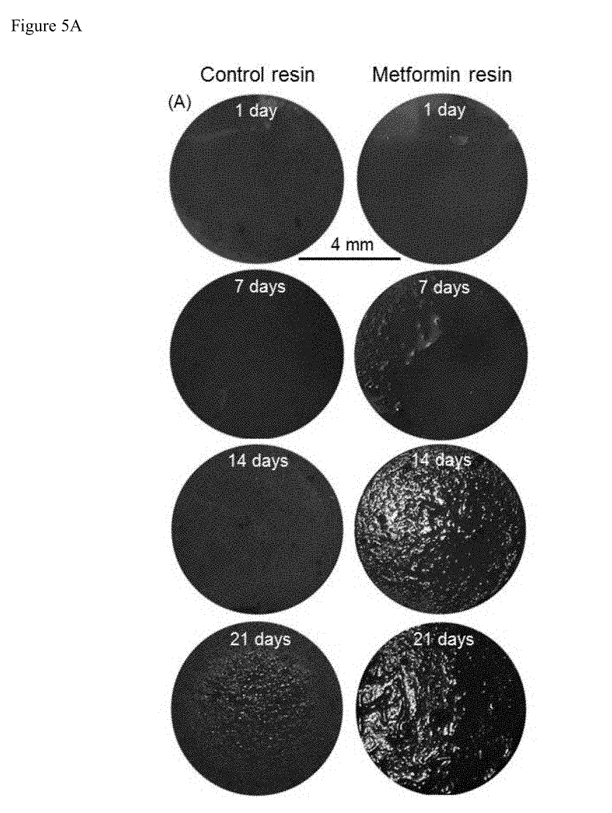

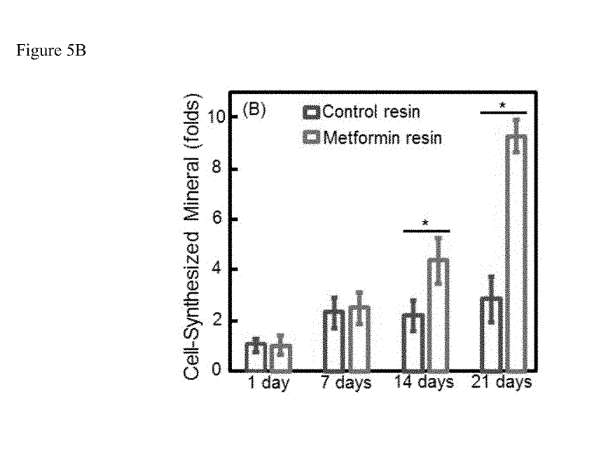

Representative Alizarin red staining (ARS) images of DPSC mineral synthesis are shown in FIG. 5A. DPSCs formed abundant mineral nodules, which were stained red. The resin disks at 1 day were stained red because of the non-specific exogenous coloring from the ARS dye; however, no mineral nodules appeared at 1 day. For resin disks with metformin, mineral nodules started to appear at 7 days and increased at 14 days; these mineral nodules showed as particle deposits on the resin which made the surface rough. With increasing time, by day 14, the disks were covered by a layer of new mineralized matrix synthesized by DPSCs, which became thicker and more abundant at day 21. In contrast, there were much less mineral nodules on control resin disks. FIG. 5B plots the quantitative mineral synthesis results (mean±SD; n=6). Mineral synthesis amount by DPSCs on metformin-resin disks greatly increased with culture time. The mineral synthesis value of metformin group at day 14 was nearly 5 folds that without metformin (p<0.05). At 21 days, mineral of metformin group was 9 folds that without metformin (p<0.05).

The results presented herein demonstrate that incorporation of metformin into a dental resin greatly enhances odontoblastic differentiation of DPSCs and increases mineral synthesis. There was no cytotoxicity from the metformin-containing resin, with the proliferation of DPSCs similar to that without metformin. Upregulation of odontoblastic gene expressions as well as mineralization-related gene expressions of DPSCs were higher on metformin resin than control resin. In addition, ALP protein synthesis by DPSCs on metformin resin was substantially enhanced. Mineral formation by DPSCs on metformin resin was 7-9 folds of that on resin without metformin. Therefore, novel metformin-containing base and liner resins for deep cavities and pulp-capping applications are promising to greatly increase tertiary dentin formation and promote pulpal repair and protection.

All documents, books, manuals, papers, patents, published patent applications, guides, abstracts and other reference materials cited herein are incorporated by reference in their entirety.

While the foregoing specification teaches the principles of the present invention, with examples provided for the purpose of illustration, it will be appreciated by one skilled in the art from reading this disclosure that various changes in form and detail can be made without departing from the true scope of the invention.

### CITED DOCUMENTS

[1] J. C. Farges, B. Alliot-Licht, E. Renard, M. Ducret, A. Gaudin, A. J. Smith, P. R. Cooper, Dental Pulp Defence and Repair Mechanisms in Dental Caries, Mediators Inflamm. 2015 (2015) 230251.
[2] J. L. Ferracane, Resin composite—state of the art, Dent. Mater. 27(1) (2011) 29-38.
[3] C. D. Lynch, R. J. McConnell, N. H. Wilson, Posterior composites: the future for restoring posterior teeth? Prim. Dent. J. 3(2) (2014) 49-53.
[4] S. Imazato, Antibacterial properties of resin composites and dentin bonding systems, Dent. Mater. 19(6) (2003) 449-57.
[5] M. S. Maas, Y. Alania, L. C. Natale, M. C. Rodrigues, D. C. Watts, R. R. Braga, Trends in restorative composites research: what is in the future? Braz. Oral Res. 31(suppl 1) (2017) e55.
[6] N. Hiraishi, C. K. Yiu, N. M. King, F. R. Tay, D. H. Pashley, Chlorhexidine release and water sorption characteristics of chlorhexidine-incorporated hydrophobic/hydrophilic resins, Dent. Mater. 24(10) (2008) 1391-9.
[7] S. Mickenautsch, V. Yengopal, A. Banerjee, Pulp response to resin-modified glass ionomer and calcium hydroxide cements in deep cavities: A quantitative systematic review, Dent. Mater. 26(8) (2010) 761-70.
[8] M. A. Saghiri, A. Asatourian, F. Garcia-Godoy, N. Sheibani, Effect of biomaterials on angiogenesis during vital pulp therapy, Dent. Mater. J. 35(5) (2016) 701-709.
[9] D. McComb, D. Ericson, Antimicrobial action of new, proprietary lining cements, J. Dent, Res. 66(5) (1987) 1025-8.
[10] I. About, P. E. Murray, J. C. Franquin, M. Remusat, A. J. Smith, The effect of cavity restoration variables on odontoblast cell numbers and dental repair, J. Dent. 29(2) (2001) 109-17.
[11] W. Zhang, P. C. Yelick, Vital pulp therapy-current progress of dental pulp regeneration and revascularization, Int. J. Dent. 2010 (2010) 856087.
[12] N. Kawashima, T. Okiji, Odontoblasts: Specialized hard-tissue-forming cells in the dentin-pulp complex, Congenit. Anom. (Kyoto) 56(4) (2016) 144-53.
[13] P. E. Murray, I. About, P. J. Lumley, J. C. Franquin, M. Remusat, A. J. Smith, Human odontoblast cell numbers after dental injury, J. Dent. 28(4) (2000) 277-85.
[14] P. E. Murray, I. About, P. J. Lumley, J. C. Franquin, M. Remusat, A. J. Smith, Cavity remaining dentin thickness and pulpal activity, Am. J. Dent. 15(1) (2002) 41-6.
[15] N. Umemura, E. Ohkoshi, M. Tajima, H. Kikuchi, T. Katayama, H. Sakagami, Hyaluronan induces odontoblastic differentiation of dental pulp stem cells via CD44, Stem Cell Res. Ther. 7(1) (2016) 135.
[16] O. Tecles, P. Laurent, S. Zygouritsas, A. S. Burger, J. Camps, J. Dejou, I. About, Activation of human dental pulp progenitor/stem cells in response to odontoblast injury, Arch. Oral Biol. 50(2) (2005) 103-8.
[17] B. Viollet, B. Guigas, N. Sanz Garcia, J. Leclerc, M. Foretz, F. Andreelli, Cellular and molecular mechanisms of metformin: an overview, Clin. Sci. (Lond) 122(6) (2012) 253-70.
[18] H. Wu, E. Esteve, V. Tremaroli, M. T. Khan, R. Caesar, L. Manneras-Holm, M. Stahlman, L. M. Olsson, M. Serino, M. Planas-Felix, G Xifra, J. M. Mercader, D. Torrents, R. Burcelin, W. Ricart, R. Perkins, J. M. Fernandez-Real, F. Backhed, Metformin alters the gut microbiome of individuals with treatment-naive type 2 diabetes, contributing to the therapeutic effects of the drug, Nat. Med. 23(7) (2017) 850-858.
[19] W. G Jang, E. J. Kim, I. H. Bae, K. N. Lee, Y. D. Kim, D. K. Kim, S. H. Kim, C. H. Lee, R. T. Franceschi, H. S. Choi, J. T. Koh, Metformin induces osteoblast differentiation via orphan nuclear receptor SHP-mediated transactivation of Runx2, Bone 48(4) (2011) 885-93.
[20] Y. Gao, J. Xue, X. Li, Y. Jia, J. Hu, Metformin regulates osteoblast and adipocyte differentiation of rat mesenchymal stem cells, J. Pharm. Pharmacol. 60(12) (2008) 1695-700.
[21] A. M. Cortizo, C. Sedlinsky, A. D. McCarthy, A. Blanco, L. Schurman, Osteogenic actions of the anti-diabetic drug metformin on osteoblasts in culture, Eur. J. Pharmacol. 536(1-2) (2006) 38-46.
[22] M. S. Molinuevo, L. Schurman, A. D. McCarthy, A. M. Cortizo, M. J. Tolosa, M. V. Gangoiti, V. Arnol, C. Sedlinsky, Effect of metformin on bone marrow progenitor cell differentiation: in vivo and in vitro studies, J. Bone Miner. Res. 25(2) (2010) 211-21.
[23] I. Kanazawa, T. Yamaguchi, S. Yano, M. Yamauchi, T. Sugimoto, Metformin enhances the differentiation and mineralization of osteoblastic MC3T3-E1 cells via AMP kinase activation as well as eNOS and BMP-2 expression, Biochem. Biophys. Res. Commun. 375(3) (2008) 414-9.
[24] P. Wang, T. Ma, D. Guo, K. Hu, Y Shu, H. H. K. Xu, A. Schneider, Metformin induces osteoblastic differentiation of human induced pluripotent stem cell-derived mesenchymal stem cells, J. Tissue. Eng. Regen. Med. 2017 May 11. doi: 10.1002/term.2470.
[25] S. Gronthos, M. Mankani, J. Brahim, P. G Robey, S. Shi, Postnatal human dental pulp stem cells (DPSCs) in vitro and in vivo, Proc. Natl. Acad. Sci. U.S.A 97(25) (2000) 13625-30.
[26] M. Yan, Y. Yu, G Zhang, C. Tang, J. Yu, A journey from dental pulp stem cells to a bio-tooth, Stem Cell Rev. 7(1) (2011) 161-71.
[27] M. K. Kolar, V. N. Itte, P. J. Kingham, The neurotrophic effects of different human dental mesenchymal stem cells, Sci. Rep. 7(1) (2017) 12605.
[28] W. Qin, X. Gao, T. Ma, M. D. Weir, J. Zou, B. Song, Z. Lin, A. Schneider, H. H. Xu, Metformin enhances the differentiation of dental pulp stem cells into odontoblasts in vitro by activating AMPK signaling, J. Endodontics 2017, in review.
[29] L. Zhang, M. D. Weir, G Hack, A. F. Fouad, H. H. Xu, Rechargeable dental adhesive with calcium phosphate nanoparticles for long-term ion release, J. Dent. 43(12) (2015) 1587-95.
[30] L. Zhang, M. D. Weir, L. C. Chow, J. M. Antonucci, J. Chen, H. H. Xu, Novel rechargeable calcium phosphate dental nanocomposite, Dent. Mater. 32(2) (2016) 285-93.
[31] S. Venz, B. Dickens, Modified surface-active monomers for adhesive bonding to dentin, J. Dent. Res. 72(3) (1993) 582-6.
[32] S. Tauscher, J. Angermann, Y. Catel, N. Moszner, Evaluation of alternative monomers to HEMA for dental applications, Dent. Mater. 33(7) (2017) 857-865.
[33] L. Wang, C. Zhang, C. Li, M. D. Weir, P. Wang, M. A. Reynolds, L. Zhao, H. H. Xu, Injectable calcium phosphate with hydrogel fibers encapsulating induced pluripotent, dental pulp and bone marrow stem cells for bone repair, Mater. Sci. Eng. C Mater. Biol. Appl. 69 (2016) 1125-36.
[34] D. J. Alongi, T. Yamaza, Y. Song, A. F. Fouad, E. E. Romberg, S. Shi, R. S. Tuan, G. T. Huang, Stem/progenitor cells from inflamed human dental pulp retain tissue regeneration potential, Regen. Med. 5(4) (2010) 617-31.
[35] N. Ayobian-Markazi, T. Fourootan, M. J. Kharazifar, Comparison of cell viability and morphology of a human osteoblast-like cell line (SaOS-2) seeded on various bone substitute materials: An in vitro study, Dent. Res. J. (Isfahan) 9(1) (2012) 86-92.
[36] W. Chen, H. Zhou, M. D. Weir, C. Bao, H. H. Xu, Umbilical cord stem cells released from alginate-fibrin microbeads inside macroporous and biofunctionalized calcium phosphate cement for bone regeneration, Acta Biomater. 8(6) (2012) 2297-306.

1. A metformin-containing dental material comprising (i) metformin in an amount of about 0.01% to about 40% of the mass of the dental material and (ii) one or more resins in a combined amount of about 20% to about 99.99% of the mass of the dental material.

2. The metformin-containing dental material of claim 1, wherein the material is a metformin-containing dental resin, metformin-containing dental adhesive, metformin-containing dental cement, metformin-containing dental composite, metformin-containing dental primer, metformin-containing dental sealant, metformin-containing dental base, metformin-containing dental coating, metformin-containing dental liner or metformin-containing dental bonding system.

3. The metformin-containing dental material of claim 2, wherein the dental material is a metformin-containing dental resin.

4. The metformin-containing dental material of claim 2, wherein the dental material is a metformin-containing dental adhesive or metformin-containing dental cement comprising (i) metformin in an amount of about 0.01% to about 40% of the mass of the adhesive or cement, (ii) one or more resins in a combined amount of about 20% to about 99.99% of the mass of the adhesive or cement, and (iii) one or more curing agents in a combined amount of about 0.05% to about 5% of the mass of the adhesive or cement.

5. The metformin-containing dental material of claim 2, wherein the dental material is a metformin-containing dental composite comprising (i) metformin in an amount of about 0.01% to about 40% of the mass of the dental composite, (ii) one or more resins in a combined amount of about 5% to about 60% of the mass of the dental composite, and (iii) one or more fillers in a combined amount of about 30% to about 90% of the mass of the composite.

6. The metformin-containing dental material according to claim 1, wherein the one or more resins are selected from the group consisting of bisphenol glycidyl methacrylate (bis-GMA), triethylene glycol dimethacrylate (TEGDMA), 2-hydroxyethyl methacrylate (HEMA), urethane dimethacrylate (UDMA), pyromellitic acid glycerol dimethacrylate (PMGDM), ethoxylated bisphenol A dimethacrylate (EBPADMA), Bis[2-(methacryloyloxy)ethyl] phosphate (BisMEP), methacryloyloxyethyl phthalate (MEP), methacrylate-modified polyalkenoic acid, pyromellitic dimethacrylate (PMDM), glycerol dimethacrylate/maleate adduct, glycerol dimethacrylate/succinate adduct, 2-acetoacetoxyethyl methacrylate, methacryloyloxyethyl maleate, a hydrophobic monomer, a hydrophilic monomer, a poly acid-modified polymer, a low shrinkage resin, a non-shrinkage resin, a light-cured polymer, a self-cured polymer, a duel cured polymer, and a heat-cured polymer.

7. The metformin-containing dental material according to claim 4, wherein the one or more curing agents are selected from the group consisting of camphorquinone (CQ), ethyl 4-N,N-dimethylaminobenzoate, phenylbis (2,4,6-triemthylbenzoyl) phosphine oxide (BAPO), and benzoyl peroxide (BPO).

8. The metformin-containing dental material according to claim 5, wherein the filler comprises one or more of a glass filler, a ceramic filler, and a polymer-based filler.

9. The metformin-containing dental material according to claim 8, wherein the filler is one or more glass fillers selected from the group consisting of barium boroaluminosilicate glass particles, fluoroaluminosilicate glass particles, fluoroaluminosilicate glass particles modified with a polyalkenoic acid, strontium-alumino-fluoro-silicate glass particles, silicon dioxide particles, fluoroaluminosilicate glass particles, fluoroaluminosilicate glass particles modified with a polycarboxylic acid, ytterbium tri-fluoride particles, and a fiber glass particles.

10. The metformin-containing dental material according to claim 8, wherein the filler is one or more ceramic fillers selected from the group consisting of a porcelain filler, a quartz filler, and a zirconia filler.

11. The metformin-containing dental material according to claim 1, further comprising one or more antibacterial agent selected from the group consisting of antibacterial monomers, quaternary ammonium salts (QASs), quaternary ammonium monomers (QAMs), silver-containing nanoparticles (NanoAgs), chlorhexidine particles, TiO₂ particles, and ZnO particles, wherein the combined amount of the one or more antibacterial agents in the dental material is about 0.5% to about 50% of the mass of the dental material.

12. The metformin-containing dental material according to claim 11, wherein the antibacterial agent is one or more antibacterial monomers,
wherein the antibacterial monomers are selected from the group consisting of dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM), and
wherein the combined amount of antibacterial monomers in the dental material is about 0.5% to about 50% of the mass of the dental material.

13. The metformin-containing dental material according to claim 11, wherein the antibacterial agent is one or more quaternary ammonium salts, and wherein the amount of quaternary ammonium salts in the dental material is about 0.01% to about 30% of the mass of the dental material.

14. The metformin-containing dental material according to claim 11, wherein the antibacterial agent is one or more silver-containing nanoparticles, and wherein the amount of silver-containing nanoparticles in the dental material is about 0.01% to about 20% of the mass of the dental material.

15. The metformin-containing dental material according to claim 1, further comprising particles of amorphous calcium phosphate (NACP) as a remineralizing agent in an amount of about 1% and 80% of the mass of the dental material.

16. The metformin-containing dental material according to claim 15, wherein the NACP particles range in size from about 10 nm to about 500 nm.

17. The metformin-containing dental material according to claim 1, further comprising one or more of acidic methacrylate or acrylate-based monomers in an amount of about 1% to about 50% of the mass of the dental material.

18. The metformin-containing dental material according to claim 1, further comprising one or more protein repellant materials in an amount of about 0.5% to about 50% of the mass of the dental material.

19. The metformin-containing dental material according to claim 18, wherein protein repellant materials are selected from the group consisting of 2-methacryloyloxyethyl phosphorylcholine (MPC), poly(hydroxyethyl methacrylate) (HEMA) and derivatives thereof, and poly(N-isopropylacrylamide) and derivatives thereof.

20. The metformin-containing dental material according to claim 1, wherein the dental material is used inside a tooth cavity, under a tooth cavity restoration, on a tooth surface, on a tooth root surface, in a periodontal pocket, or in a root canal.
